(19) United States
(12) Patent Application Publication
(10) Pub. No.: US 2021/0319858 A1
(43) Pub. Date: Oct. 14, 2021

(54) ASSIGNING MEDICAL CODINGS

(71) Applicant: INTERNATIONAL BUSINESS MACHINES CORPORATION, Armonk, NY (US)

(72) Inventors: Matthias Reumann, Herrenberg (DE); Andrea Giovannini, Zurich (CH)

(21) Appl. No.: 16/848,029

(22) Filed: Apr. 14, 2020

(51) Int. Cl. G16H 10/60; G16H 15/00; G16H 50/20; G16H 30/40; G16H 30/20; G16H 40/63; G16H 40/67; G06F 40/40; G06N 3/02

(52) U.S. Cl. CPC G16H 10/60; G16H 15/00; G16H 50/20; G16H 30/40; G06N 3/02; G16H 40/63; G16H 40/67; G06F 40/40; G16H 30/20

(57) ABSTRACT

The exemplary embodiments disclose a system and method, a computer program product, and a computer system for assigning medical codes. The exemplary embodiments may include receiving a medical record comprising a diagnosis code, querying a knowledge graph using the diagnosis code, the knowledge graph comprising as nodes: case identifiers, diagnosis codes and related procedure codes, and secondary diagnosis codes and related secondary procedure codes, wherein edges between the nodes are indicative of a type of relationship between related nodes derived from real past medical records, and receiving, based on the query, a ranked list of the diagnosis codes, related procedure codes, and secondary diagnosis codes and the related secondary procedure codes based on relative occurrences of the past medical records.

### BACKGROUND

The invention relates generally to identifying treatment codes in medical records, and more specifically, to a computer-implemented method for assigning medical codes. The invention relates further to a coding system for assigning medical coding, a computing system, and a computer program product.

National and private healthcare systems around the world are supporting increasingly complex and expensive treatments. In any case, the insurance companies bear the burden of paying the amount paid to hospitals and doctors. Technically, however, there is no closed information supply chain from diagnosis through one or more treatments, which are usually part of a frequently handwritten medical record, to insurance companies.

Thus, medical coding of diagnoses and procedures shall determine a reimbursement for medical services in a consolidated and reliable way. The market for systems for medical coding is about US$17 billion worldwide in 2015 and US$1.25 billion in Germany alone. However, medical coding is cumbersome and manual process with the following pain points. One side of the coin is that not enough qualified personnel is available for the ever-growing complexity of medical diagnosis and treatment and related payments. In other words, there is a significant lack of expertise. On the other side, even if specialists would be available for, e.g., a hospital, during sickness or vacation of these experts, the revenue of a hospital is directly negative affected. As a third aspect, up to now, human expertise continues to be a key ingredient for an optimal medical coding. Inexperienced coders make mistakes, make wrong medical code assignments, and cause additional costs for the hospitals and/or insurance companies.

Currently, there are some products on the market that try to provide medical coding support by providing search capabilities for a code catalog and some statistical methods to provide probabilities of possible medical codes related to what a coder has already defined. However, none of these techniques solve the issue of the manual process the coder has to go through to code a medical case completely and correctly. Simple search engines do not provide the information capable of capturing the details required for a full coding support, even not with natural language processing methodologies.

### SUMMARY

The exemplary embodiments disclose a system and method, a computer program product, and a computer system for assigning medical codes. The exemplary embodiments may include receiving a medical record comprising a diagnosis code, querying a knowledge graph using the diagnosis code, the knowledge graph comprising as nodes: case identifiers, diagnosis codes and related procedure codes, and secondary diagnosis codes and related secondary procedure codes, wherein edges between the nodes are indicative of a type of relationship between related nodes derived from real past medical records, and receiving, based on the query, a ranked list of the diagnosis codes, related procedure codes, and secondary diagnosis codes and the related secondary procedure codes based on relative occurrences of the past medical records.

### BRIEF DESCRIPTION OF THE SEVERAL VIEWS OF THE DRAWINGS

It should be noted that embodiments of the invention are described with reference to different subject-matters. In particular, some embodiments are described with reference to method type claims, whereas other embodiments are described with reference to apparatus type claims. However, a person skilled in the art will gather from the above and the following description that, unless otherwise notified, in addition to any combination of features belonging to one type of subject-matter, also any combination between features relating to different subject-matters, in particular, between features of the method type claims, and features of the apparatus type claims, is considered as to be disclosed within this document.

The following detailed description, given by way of example and not intended to limit the exemplary embodiments solely thereto, will best be appreciated in conjunction with the accompanying drawings, in which:

FIG. 1 shows a block diagram of an embodiment of the inventive computer-implemented method for assigning medical codes.

FIG. 2 shows a block diagram of an embodiment of building the knowledge graph for the proposed concept.

FIG. 3 shows an exemplary diagram of a small, but representative part of the knowledge graph.

FIG. 4 shows a block diagram of a flowchart embodiment of how the proposed concept may work in operation mode.

FIG. 5 shows an exemplary diagram of the operational mode of the proposed concept under a different view.

FIG. 6 shows a fragment of pseudo-code for the main activity described in the context of FIG. 5.

FIG. 7 shows a block diagram of the proposed coding system.

FIG. 8 depicts an exemplary block diagram depicting the hardware components of the disclosed system, in accordance with the exemplary embodiments.

FIG. 9 depicts a cloud computing environment, in accordance with the exemplary embodiments.

FIG. 10 depicts abstraction model layers, in accordance with the exemplary embodiments.

The drawings are not necessarily to scale. The drawings are merely schematic representations, not intended to portray specific parameters of the exemplary embodiments. The drawings are intended to depict only typical exemplary embodiments. In the drawings, like numbering represents like elements.

### DETAILED DESCRIPTION OF THE SEVERAL VIEWS OF THE DRAWINGS

In the context of this description, the following conventions, terms and/or expressions may be used:

The term ‘medical coding’ may denote a process of assigning appropriate medical codes to medical terms in patient records. Additionally, medical coding may also be directed to assigning treatment codes of one or more medical treatments relating to a medical diagnosis code.

The term ‘medical record’ may denote an entry in a patient's file, wherein an individual record relates to one or more diagnoses and optionally also to medical treatments. Medical records are still very often only available in hand-written forms. Another hurdle is that often hospital specific terms are used so that it is technically difficult to bridge the gap from individual hospitals with “local diagnoses and local treatments” to insurance companies. In case, the handwritten forms of the medical records have been “electronified” they are typically available in PDF (portable document format) form or as simple digital image.

The term ‘diagnosis code’ may denote an entry in a standardized domain specific code table for medical diagnoses. A typical example is the ICD-10 code (International Classification of Diseases) or, the other side, the medical treatment code system, namely the procedure code system(s).

The term ‘procedure code’ may denote one of a catalog of standardized medical treatments. One example of such a catalog, often used in Germany, is denoted OPS (operations and procedure code); another one is CHOP, the Swiss catalog of operations and procedures. Other countries may have other catalogs in their respective medical systems. Additionally, in this context, the term ‘DRG handbook’ may be defined: diagnosis related groups. Groups may denote groups of procedures relatable to a specific diagnosis. However, there is not a direct 1:1 relationship between a medical diagnosis and one or more related procedures. The medical world is much more complex, treatments change over time and may also be hospital or doctor specific, and may underlie regional and medical school specific variations.

The term ‘case identifiers’—in short caseID—may denote a unique code instrumental for identifying a specific diagnosis for a specific patient. The case identifier may represent the non-interchangeable code identifying a specific patient, a diagnosis time, the diagnosis and optionally prescribed medical procedures.

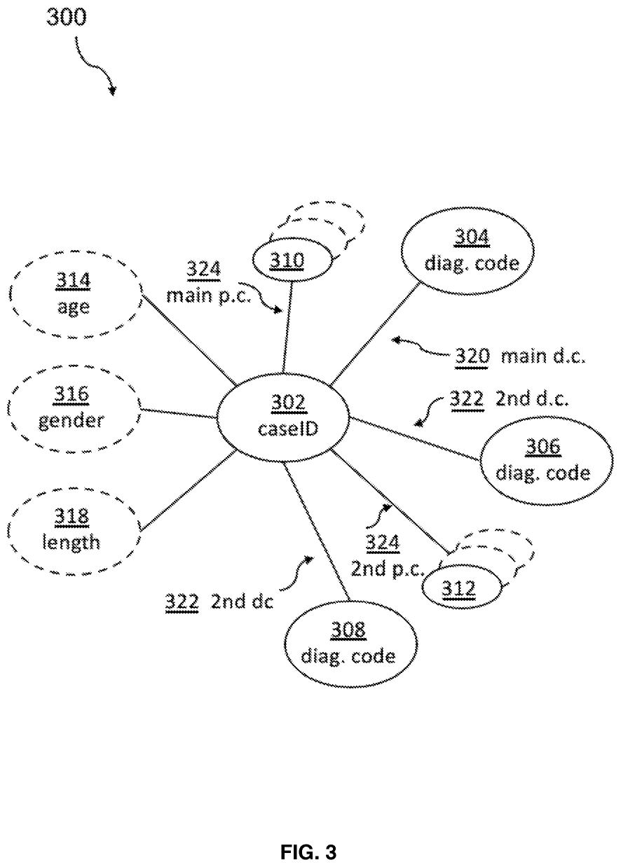

The term ‘knowledge graph’ may denote, in a broad sense, a database for storing information of different topics and dynamically relating single expressions to each other. Knowledge graphs may be well suited for finding specific information and relating different terms/expressions to each other. For that, often distance measures are used between terms stored in the knowledge graph. Typically, expressions or terms of facts are stored in ‘nodes’ in a knowledge graph, while connections between individual ones of the nodes are termed ‘edges’. These edges may also have attributes, e.g., describing the relationship between the nodes an edge may be connected to. In other cases, the edges may carry weights expressing a relationship strength between the connected nodes. A small example is described below in the context of FIG. 3.

The term ‘real past medical record’ may denote a medical record of a real case of a patient comprising already at least one correctly coded diagnosis code in combination with at least one procedure code. From these real past medical records, the knowledge graph as decision basis may be constructed. Additionally, the complete knowledge graph decision basis for correctly assigning medical diagnosis codes and procedure codes may incorporate a larger plurality of information sources.

The term ‘optical character recognition’—abbreviated as OCR—may denote a technique to translate hand-written letters or expressions into a machine readable form. For this, image recognition techniques may be used which may be supported by natural language processing (NLP) systems being trained for classifying terms and expressions in the medical records, as well as dependencies and typical sequences of expressions. Also, other machine-learning techniques, like neural networks, may be supportive for the here proposed concept.

According to one aspect of the present invention, a computer-implemented method for assigning medical codes may be provided. The method may comprise receiving a medical record comprising a diagnosis code, and querying a knowledge graph using the diagnosis code, the knowledge graph comprising as nodes: case identifiers, diagnosis codes and related procedure codes, secondary diagnosis codes and related secondary procedure codes, wherein edges between the nodes are indicative of a type of relationship between related nodes derived from real past medical records.

Additionally, the method may comprise receiving, based on the query, a ranked list of the diagnosis codes, related procedure codes, and secondary diagnosis codes and related secondary procedure codes, based on relative occurrences of the past medical records.

According to another aspect of the present invention, a coding system for assigning medical codes may be provided. The coding system may comprise means for receiving a medical record comprising a diagnosis code, means for querying a knowledge graph using the diagnosis code, the knowledge graph comprising as nodes: case identifiers, diagnosis codes and related procedure codes, secondary diagnosis codes and related secondary procedure codes, wherein edges between the nodes are indicative of a type of relationship between related nodes which may be derived from real past medical records, and means for receiving, based on the query, a ranked list of the diagnosis codes, related procedure codes, secondary diagnosis codes and related secondary procedure codes based on relative occurrences derived of the past medical records.

The proposed computer-implemented method for assigning medical coding may offer multiple advantages, technical effects, contributions and/or improvements:

Essentially, the proposed concept discloses the feature of querying a knowledge graph for retrieving main procedural codes, ranking the returned list of main procedural codes using statistics of the historical data, and enables an automatic coding of the medical case if a predefined threshold for relationship probabilities between diagnosis codes and procedure codes is exceeded.

A combination of different machine-learning methods and/or systems may be used for enabling a relationship building between diagnosis codes and procedural codes. Firstly, handwritten medical records may be scanned and treated with OCR techniques in order to identify diagnosis codes. This process may be supported by NLP (natural language processing) techniques in order to isolate the medical codes from the context it is embedded in.

Furthermore, a knowledge graph may be used, in which information from a plurality of sources may be stored, to build a complex network of nodes and relationships, thereby delivering a sorted list of potential paths of medical diagnosis codes and related procedural codes—in particular, main and/or secondary diagnosis codes, as well as, main procedure codes, as well as, secondary procedure codes—as a result of a query. Thereby, the sorting of the list may be influenced by those medical cases with which the knowledge graph has been constructed. This may be hospital specific and/or insurance specific, depending on the medical records the knowledge graph has been built with. In addition to the ground truth of past real medical cases and its related and existing medical coding, complete catalogs of diagnosis codes and treatment procedure codes of different sources may also be used as further input to build the knowledge graph. Moreover, also rule of thumb guidelines—like the DRG handbook—may be used to enhance the content of the knowledge graph and qualify the edges, i.e., assign attributes to the edges of the knowledge graph.

Furthermore, the already mentioned NLP system or another, second NLP system trained according to different expressions and interdependencies between expressions may be used in order to verify records of the output of the query to the knowledge graph (the ranked list) in order to identify specific evidence in the medical records to confirm (or not) an output of the query—i.e., the ranks in the list—to the knowledge graph. According to the evidence value, the ranks in the list may also be changed such that the most probable relationships between diagnosis codes and procedure codes may be at the beginning of the list.

It may turn out that a high probability exists—i.e., a probability value above a predefined threshold value—for a pair of a main diagnosis code and a related main procedure code and/or secondary diagnosis codes and related secondary procedure code(s), the medical coding, i.e., the assignment of procedural codes to diagnosis codes, may be made automatically and without human intervention. However, there may also exist the option to ask for a confirmation by a human operator for the identified pairs of diagnosis codes and procedural codes. This can be implementation specific.

It may be noted that the proposed concept may not only be directed to medical coding but may also be applicable to other domain specific measurement/response relationships. One example may be a quality control system in a production environment. In that case, the diagnosis codes may relate to output codes (e.g., error classifications) of a quality measurement system (e.g., surface smoothness of a produced device, correctness of a color code, just to name a few), and the procedural codes may relate to treatments to the measured device of produced part in order to either enhance their quality to bring it in line with specifications or to separate the produced part from the production line. There may be more domain specific applications for the here proposed concept.

In the following, additional embodiments of the inventive concept—applicable for the method as well as for the related system—will be described.

According to a useful embodiment, the method may also comprise scanning the medical record, and extracting the diagnosis code using optical character recognition (OCR). In some cases, also other information, e.g., other diagnosis codes, i.e., secondary diagnosis codes may be extracted from the medical record. It may also be mentioned that the diagnosis code that is extracted from the medical record may be seen as the main diagnosis code. The used OCR system may, in particular, be trained for medical expressions typically used in medical records, and it may be supported or be based on a machine-learning system.

Consequently, according to another embodiment of the method, the medical record may also comprise a secondary diagnosis code—in particular, at least one—with a related secondary procedure code. Also, for the main diagnosis code, a main procedure code may be extractable from the medical record.

According to a further embodiment, the method may also comprise extracting from the scanned medical record a procedure code relating to the diagnosis code, and at least one secondary diagnosis code with the related secondary procedure code using a trained natural language processing system. Hence, using the OCR system, one may try to extract as much information as possible, in particular, in the form of structured medical codes and sub-codes.

According to one advantageous embodiment of the method, the knowledge graph may comprise as additional nodes an age, gender information, and/or length of stay, i.e., hospital stay, of a patient. Also, other relevant patient data may be stored as part of the knowledge graph. These additional data may help to identify the most relevant procedure codes for a given case, i.e., a given case identifier used as key for the medical record.

According to a further advantageous embodiment, the method may also comprise displaying the ranked list, in particular according to a predefined format, and receiving a confirmation signal—in particular from a user—for a selected combination of the diagnosis code and one or more related procedure codes. This way, the machine and knowledge based analytic result, the recommendation and the so-far automatic decision/assignment process may get a final confirmation from an experienced user.

The so generated additional medical code records with structured medical codes and relationships, i.e., main diagnosis codes, secondary diagnoses codes, main procedure codes, as well as secondary procedure codes, may be used for a subsequent enhancement of the knowledge graph. The so generated additional data may be stored in the knowledge graph on a continuous basis, or if a predefined number of additional medical records have been processed. This way, the self-learning process may be initiated allowing an even better recognition, processing and assigning of data in medical records. This way, it becomes more and more likely that no manual confirmation step is required, and that increasingly automatic data assignments may be performed.

However, as long as the just-described approach is not possible and according to a further possible embodiment, the method may also comprise receiving a confirmation signal for a selected combination of at least one second diagnosis code and one or more related procedure codes. These selected possible combinations have been rendered on a display in a predefined manner allowing an easy and fast confirmation by a user, e.g., using checkmark boxes.

According to one additionally advantageous embodiment, the method may also comprise building the knowledge graph from real past medical records that have already been coded manually and correctly, and a catalogue of diagnosis codes—in particular, ICD-10 codes and procedure codes—in particular, CHOP and/or/OPS codes—and/or rules for dependencies between diagnosis codes and procedure codes, e.g., based on the DRG handbook. This embodiment may be seen as a useful preparatory step of the main method in order to enable an execution of the main method in an operational mode.

According to an enhanced embodiment, the method may also comprise confirming directly and automatically, upon determining, in the ranked list, that a predefined percentage of the selected diagnosis code relate to a specific procedure code, a relationship between the diagnosis code and the procedure code. The predefined percentage may in a specific determination step be treated as a threshold value above which the automatic direct confirmation may be performed. Thus, no human intervention may be required in order to build the right pairs of diagnostic codes and procedure codes.

According to a further possible embodiment of the method, the diagnosis code may be a plurality of diagnosis codes. In such a case, a main diagnosis code and one or more secondary diagnosis codes may have been used in the medical record. The here proposed concept also works for such a multi-occurrence of diagnosis codes in the medical record. It may only be required to extend the resulting data structure of the above-mentioned OCR process to store more than one diagnoses code.

According to an even further enhanced embodiment, the method may also comprise determining an evidence factor value indicative of a probability for at least one of a main diagnosis code, any other diagnosis code—in particular a secondary diagnosis code, a main procedure code or any other—in particular his secondary—procedure code using a trained natural processing language system. The analysis of the medical record for a determination of the evidence factor value may search for specific keywords in a combination and in the context of a possible diagnosis code and/or procedural code in order to change the ranking of the resulting list such that most probable combinations of diagnosis codes and procedural codes are ranked to the beginning of the ranked list. This way, basically three different machine-learning systems may be applied to a processing of medical records: the first OCR system identifying a (main) diagnostic code in a medical record, the knowledge graph, as well as the second OCR system looking for the evidence factor value for a better and optimized sorting of the ranked list of diagnosis codes and procedure codes.

Furthermore, embodiments may take the form of a related computer program product, accessible from a computer-usable or computer-readable medium providing program code for use, by, or in connection, with a computer or any instruction execution system. For the purpose of this description, a computer-usable or computer-readable medium may be any apparatus that may contain means for storing, communicating, propagating or transporting the program for use, by, or in connection, with the instruction execution system, apparatus, or device.

In the following, a detailed description of the figures will be given. All instructions in the figures are schematic. Firstly, a block diagram of an embodiment of the inventive computer-implemented method for assigning medical coding is given. Afterwards, further embodiments, as well as embodiments of the coding system for assigning medical coding, will be described.

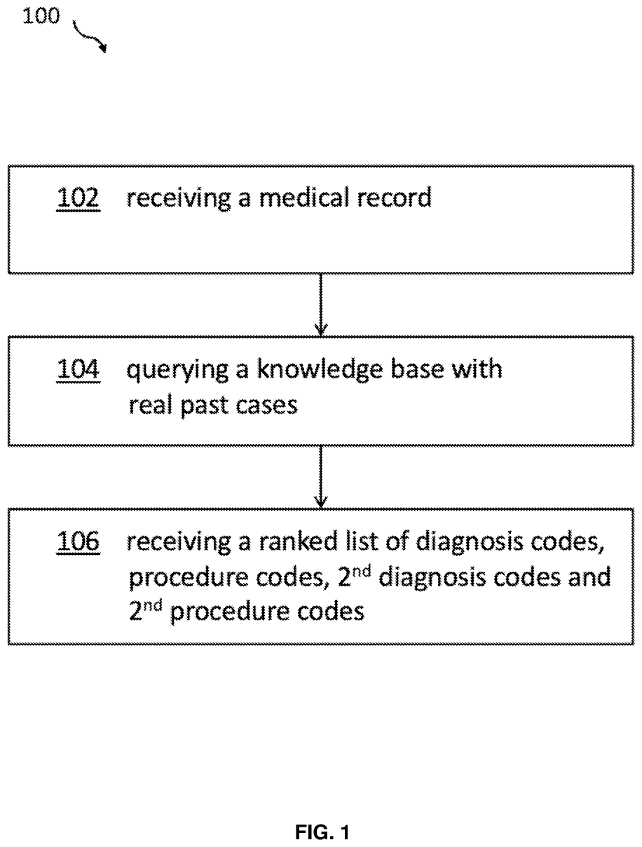

FIG. 1 shows a block diagram of a preferred embodiment of the computer-implemented method 100 for assigning medical codes. The method comprises receiving, 102, a medical record, e.g., in PDF form, digital image form or just handwritten—comprising a diagnosis code, e.g. just a main diagnosis code. Optionally, also a related main procedure code and secondary diagnosis codes together with the related secondary procedure codes may be extractable from the medical record.

Additionally, the method 100 comprises querying, 104, a knowledge graph using at least the diagnosis code as input. Thereby, the knowledge graph comprises as nodes case identifiers, diagnosis codes and related procedure codes, and secondary diagnosis codes and related secondary procedure codes. The edges between the nodes are indicative of a type of relationship between related nodes. A case identifier is typically being related to a main diagnosis code; thus, the edge carries the attribute “main diagnosis”. However, the case identifier may also relate to secondary diagnosis code(s). In that case, the connecting action between the case identifier and one of the secondary diagnosis codes carries the attribute “secondary diagnosis”. This system should be understood to be used for most relationships within the knowledge graph. On the other side, there may be nodes relating to a case identifier that, by its nature, specifies a predefined relationship. If the node type is, e.g., of type “age”, the edge is not required to carry an additional attribute because the type of node—here, “age”-specifies the relationship in a unique way. Node types like “gender” and “length of stay”—i.e., a length of a stay of a patient in the hospital or in a specific department of a hospital—are treated similarly.

Furthermore, the method 100 comprises receiving, 106, based on the query, a ranked list of the diagnosis codes, related procedure codes, secondary diagnosis codes and related secondary procedure codes based on relative occurrences of the past medical records.

This way, the proposed concept supports the potential to completely automate the coding process of diagnosis and related procedures.

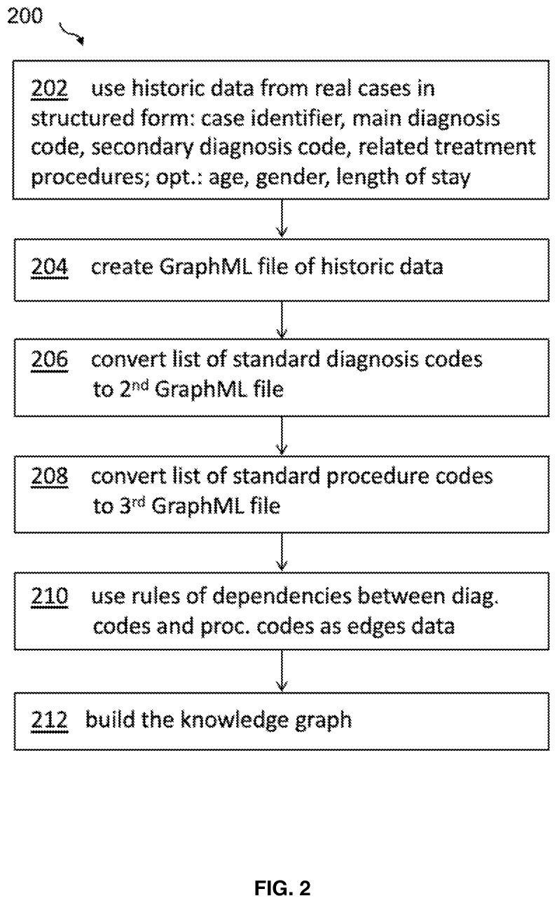

FIG. 2 shows a block diagram 200 of an embodiment of building the knowledge graph for the proposed concept. As a first step, historic data from real medical cases—i.e., medical records—may be made available in structured form, 202. Hence, these data have undergone a medical coding (by a human) already. In the best case, these historic data from real medical cases comprise a main diagnosis code, one or more secondary diagnosis codes and related treatment codes. Optionally, also the age of the patient, their gender, and the length of their stay in a hospital or a department of a hospital may also be used. Based on this—in one embodiment—a first graphML file is built, 204. GraphML is known as an XML-based file from what to describe or define knowledge graphs.

Secondly, a list of standard diagnosis codes is also converted, 206, into a second graphML file. Furthermore, a known list of standard procedure codes is also converted, 208, into a third graphML file. In combination, the first, the second and the third graphML file are used to define and specify the knowledge graph.

Furthermore, also rules of dependencies between diagnosis codes and procedural codes may be added, 210, to the knowledge graph as attributes of edges within the knowledge graph. Thus, a probability that a specific procedural code may relate to a specific diagnosis code may be expressed with these rules, i.e., attributes. All of the above-mentioned data are used to build, 212, the final knowledge graph which may be used as basis for queries of newly analyzed medical records.

FIG. 3 shows an exemplary diagram of a small, but representative part of the knowledge graph 300. In the center, the caseID 302 (case identifier for a specific medical case) is shown. Related to this is a diagnostic code 304 which typically has been identified by the first machine-learning based NLP system in which the diagnostic code 304 should have been identified from the digital image of a handwritten medical record. The edge 320 between the caseID node 302 and the diagnosis code 304 is shown as main diagnostic node as attribute of the edge 320. Similarly, the edge 322 may describe the diagnosis code 308 as secondary diagnosis code. Furthermore, the nodes 312 may be defined as secondary procedure codes. The same may apply to the node(s) 310 which may be defined by the edge 324 as main procedure code(s).

Additionally, other attributes of the patient, like age 314, gender 316 or length of stay 318 (and optionally additional patient attributes), can be linked via edges to the caseID 302.

Many of such a knowledge sub-graph cell (as just described) build the basis for a complete knowledge graph comprising information about many caseIDs enhanced by information from a catalog of diagnosis codes (e.g., ICD-10 codes) and procedural codes (e.g., OPS and/or CHOP codes) together with a set of rules (like the dependency rules codified and the DRG handbook).

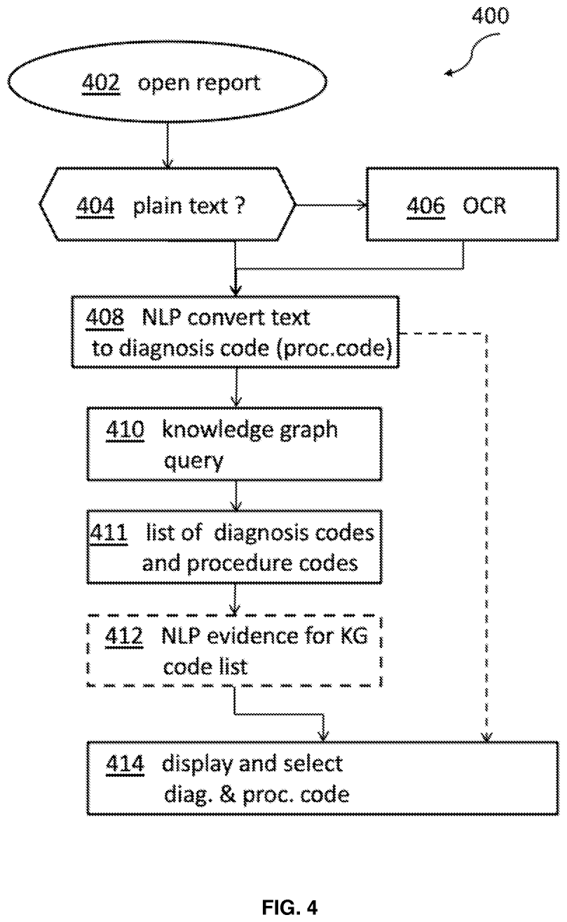

FIG. 4 shows a block diagram of a flowchart 400 of an embodiment of how the proposed concept may work in operation mode. Firstly, the medical record or report is opened, 402. If the read medical record is not available in plain and machine-readable form (determination 404) an OCR system converts, 406, the read medical record into a machine-readable format. The OCR system can be supported by an NLP system, as well as a trained machine-learning system for image recognition. These systems combined shall ensure that the information available in the medical record may at least be converted, 408, into a diagnosis code—in particular, a main diagnosis code—and optionally also a secondary diagnosis code, and also optionally, to related procedural codes.

The output from this step of recognizing, 408, at least one diagnosis code is used, 410, as query data for the knowledge graph. The knowledge graph query results, 411, in a ranked list of diagnosis codes (main and secondary) and related procedural codes (main and secondary).

As a further and optional step, the results available in the ranked list can now each undergo a specific evaluation, 412, by a second NLP system (or an enhanced version of the above-mentioned NLP system)—optionally supported by a trained related machine-learning system—to find evidence in the original underlying medical record whether the proposed relationship between a medical code and one or more procedural codes has a certain probability. These evidence values can additionally be used to influence the ranking in the ranked list which was the output of the knowledge graph. The higher a probability for a pair of diagnosis code and procedure code is, the higher such a pair is ranked in the list.

The final step shown in this flowchart relates to displaying and selecting and/or confirming (by an expert) relationships of diagnosis codes and procedural codes.—Optionally, the by evidence factor resorted ranked knowledge graph list may be compared to the originally recognized diagnosis code from step 408 (dashed line from step 408 to step 414).

A further or alternative step to the last step of displaying and selecting, 414, a diagnosis code/procedural code pair can be an automatic assignment of a diagnosis code to one or more procedural codes. This may be executed if the ranked list (eventually re-sorted by the evidence step 412) shows a probability for the relationship of the recognized diagnosis code (identified/recognized in the medical record) and the procedural code that is above a predefined threshold value.

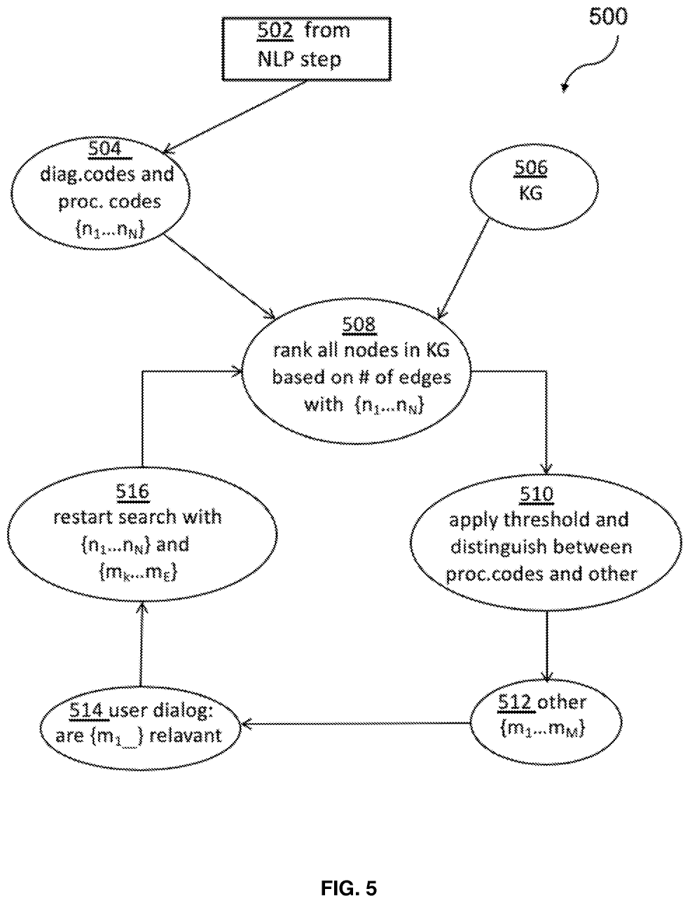

FIG. 5 shows an exemplary diagram 500 of the operational mode of the proposed concept under a different view. This diagram starts, 502, with the first NLP step (compare FIG. 4, 408). Thus, in status 504, diagnostic codes and procedural codes {n₁, . . . , n_{N}} have been identified. The knowledge graph 506 has already been built, as described above.

In the status ellipse 508, the identified nodes based on the query, coming from the status 504, are ranked as output of the knowledge graph based on the number of edges for the codes {n₁, . . . , n_{N}}. Next, 510, the above-mentioned threshold value and comparison is applied and it is distinguished between procedural codes and other information (like age, gender, length of stay).

The other information (i.e., attributes or nodes) {m₁, . . . , m_{M}} are available in the status 512. Next, a user dialogue may happen to decide whether the found mᵢ additional attributes are relevant at all for a decision for a relationship between a diagnostic code and one or more procedural codes. As a result, status ellipse 514, a list of additional attributes {mₖ, . . . , m_{E}} is available. This can also be an empty group.

Then, this search in the knowledge graph is repeated with the {n₁, . . . , n_{N}} diagnosis codes and the additional attributes {mₖ, . . . , m_{E}}, 516. Using this additional loop for reflecting also the additional attributes, the ranked list shows with a significantly higher probability the correct diagnosis code to procedure code relationship(s).

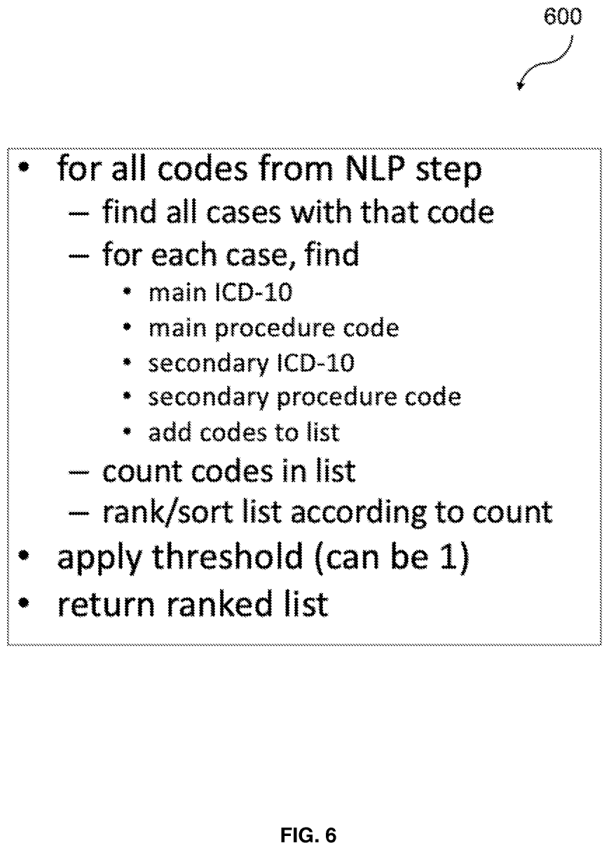

FIG. 6 shows a fragment of pseudo-code 600 for the main activity described in the context of FIG. 5, in particular to activity ellipse 508, i.e., the ranking/query step performed in combination with the knowledge graph.

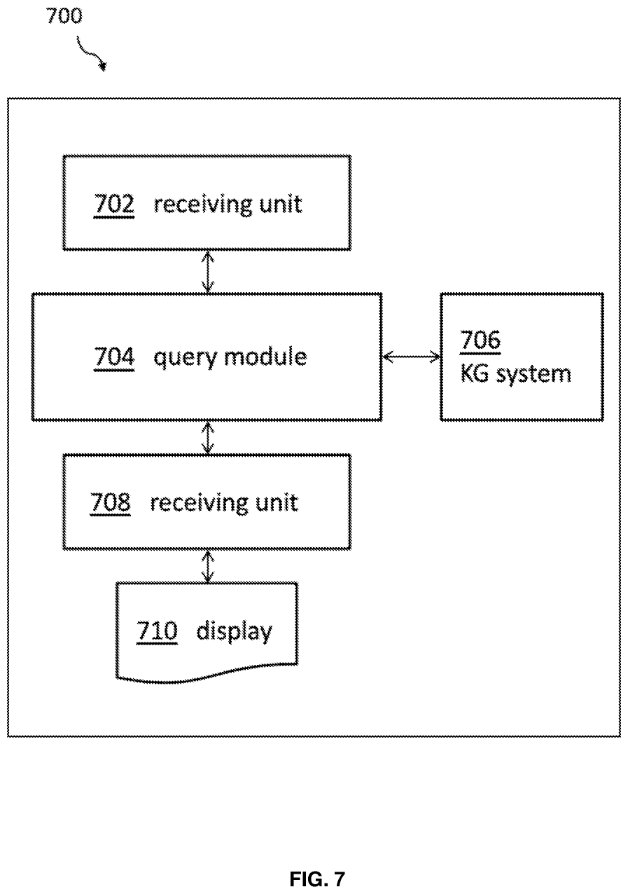

For completeness reasons, FIG. 7 shows a block diagram of the proposed coding system 700. The coding system 700 comprises means for receiving—in particular, a hardware-based receiving unit 702—a medical record comprising at the least one diagnosis code.

Additionally, the coding system 700 comprises means for querying—in particular, a hardware-based query module 704—a (hardware-implemented) knowledge graph system 706 using the diagnosis code, the knowledge graph comprising as nodes: case identifiers, diagnosis codes and related procedure codes, and secondary diagnosis codes and related secondary procedure codes, wherein edges between the nodes are indicative of a type of relationship between related nodes derived from real past medical records.

Last but not least, the coding system 700 comprises means for receiving—in particular, a second hardware-based receiving unit 708—based on the query, a ranked list of the diagnosis codes, related procedure codes, secondary diagnosis codes and related secondary procedure codes based on relative occurrences of the past medical records.

It should be noted that the receiving unit 702 is connected to the query module 704 in order to use the output of the receiving unit 702 as input to the query module 704. In order to use the output of the query module 704, it is connected to the second receiving unit 708.

Furthermore, the coding system 700 can comprise a display unit 710 showing, e.g., the results of the receiving unit 708 to which the display unit 710 is the hardware-wise connected. Thus, an interconnected basic system for the coding system 700 is provided. Also, the other above-described method steps may be implemented as hardware-based modules or units connected to the here described hardware modules or units.

Embodiments of the invention may be implemented together with virtually any type of computer, regardless of the platform being suitable for storing and/or executing program code.

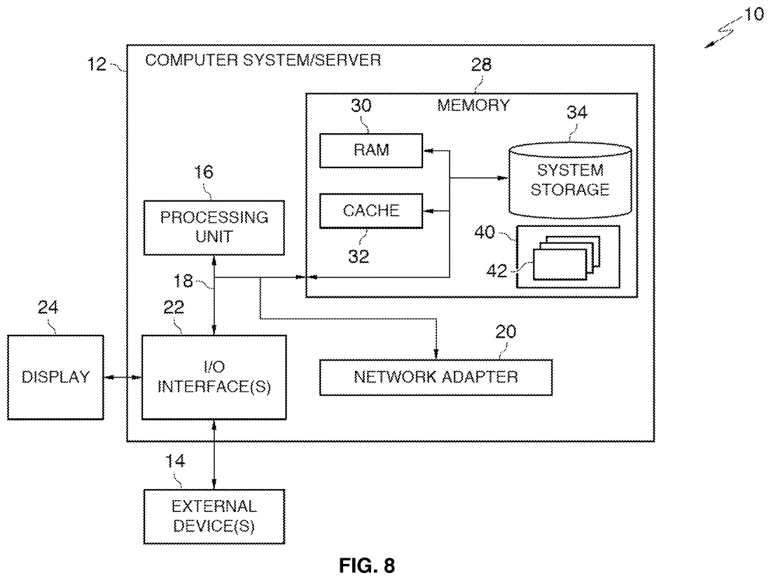

FIG. 8 depicts a block diagram of devices in accordance with the exemplary embodiments. It should be appreciated that FIG. 8 provides only an illustration of one implementation and does not imply any limitations with regard to the environments in which different embodiments may be implemented. Many modifications to the depicted environment may be made.

Devices used herein may include one or more processors 02, one or more computer-readable RAMs 04, one or more computer-readable ROMs 06, one or more computer readable storage media 08, device drivers 12, read/write drive or interface 14, network adapter or interface 16, all interconnected over a communications fabric 18. Communications fabric 18 may be implemented with any architecture designed for passing data and/or control information between processors (such as microprocessors, communications and network processors, etc.), system memory, peripheral devices, and any other hardware components within a system.

One or more operating systems 10, and one or more application programs 11 are stored on one or more of the computer readable storage media 08 for execution by one or more of the processors 02 via one or more of the respective RAMs 04 (which typically include cache memory). In the illustrated embodiment, each of the computer readable storage media 08 may be a magnetic disk storage device of an internal hard drive, CD-ROM, DVD, memory stick, magnetic tape, magnetic disk, optical disk, a semiconductor storage device such as RAM, ROM, EPROM, flash memory or any other computer-readable tangible storage device that can store a computer program and digital information.

Devices used herein may also include a R/W drive or interface 14 to read from and write to one or more portable computer readable storage media 26. Application programs 11 on said devices may be stored on one or more of the portable computer readable storage media 26, read via the respective R/W drive or interface 14 and loaded into the respective computer readable storage media 08.

Devices used herein may also include a network adapter or interface 16, such as a TCP/IP adapter card or wireless communication adapter (such as a 4G wireless communication adapter using OFDMA technology). Application programs 11 on said computing devices may be downloaded to the computing device from an external computer or external storage device via a network (for example, the Internet, a local area network or other wide area network or wireless network) and network adapter or interface 16. From the network adapter or interface 16, the programs may be loaded onto computer readable storage media 08. The network may comprise copper wires, optical fibers, wireless transmission, routers, firewalls, switches, gateway computers and/or edge servers.

Devices used herein may also include a display screen 20, a keyboard or keypad 22, and a computer mouse or touchpad 24. Device drivers 12 interface to display screen 20 for imaging, to keyboard or keypad 22, to computer mouse or touchpad 24, and/or to display screen 20 for pressure sensing of alphanumeric character entry and user selections. The device drivers 12, R/W drive or interface 14 and network adapter or interface 16 may comprise hardware and software (stored on computer readable storage media 08 and/or ROM 06).

The programs described herein are identified based upon the application for which they are implemented in a specific one of the exemplary embodiments. However, it should be appreciated that any particular program nomenclature herein is used merely for convenience, and thus the exemplary embodiments should not be limited to use solely in any specific application identified and/or implied by such nomenclature.

Based on the foregoing, a computer system, method, and computer program product have been disclosed. However, numerous modifications and substitutions can be made without deviating from the scope of the exemplary embodiments. Therefore, the exemplary embodiments have been disclosed by way of example and not limitation.

It is to be understood that although this disclosure includes a detailed description on cloud computing, implementation of the teachings recited herein are not limited to a cloud computing environment. Rather, the exemplary embodiments are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or data center).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

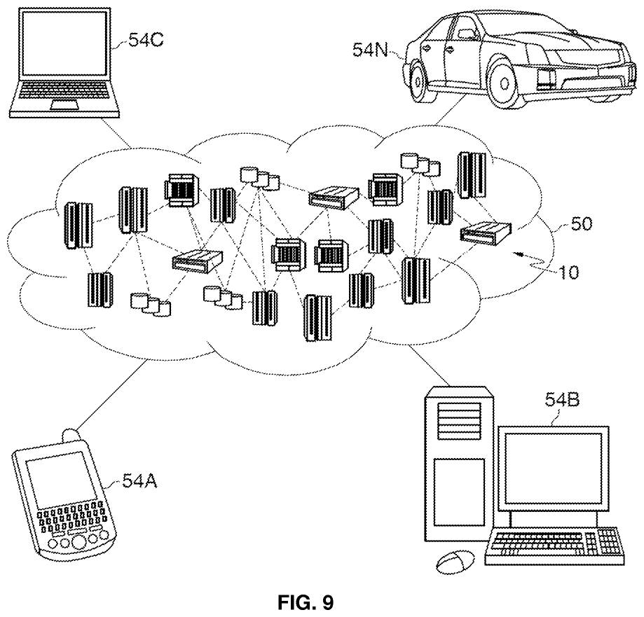

Referring now to FIG. 9, illustrative cloud computing environment 50 is depicted. As shown, cloud computing environment 50 includes one or more cloud computing nodes 40 with which local computing devices used by cloud consumers, such as, for example, personal digital assistant (PDA) or cellular telephone 54A, desktop computer 54B, laptop computer 54C, and/or automobile computer system 54N may communicate. Nodes 40 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 9 are intended to be illustrative only and that computing nodes 40 and cloud computing environment 50 can communicate with any type of computerized device over any type of network and/or network addressable connection (e.g., using a web browser).

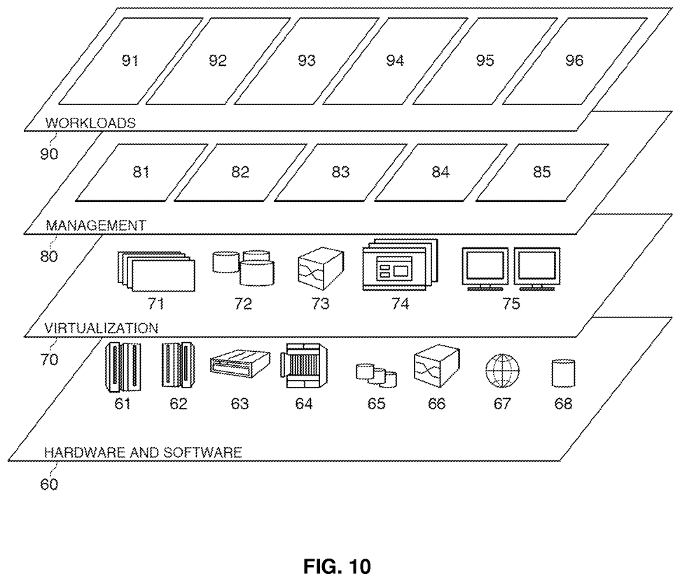

Referring now to FIG. 10, a set of functional abstraction layers provided by cloud computing environment 50 (FIG. 9) is shown. It should be understood in advance that the components, layers, and functions shown in FIG. 10 are intended to be illustrative only and the exemplary embodiments are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and the assigning of medical codings 96.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skills in the art without departing from the scope and spirit of the described embodiments. The terminology used herein was chosen to best explain the principles of the embodiments, the practical application or technical improvement over technologies found in the marketplace, or to enable others of ordinary skills in the art to understand the embodiments disclosed herein.

The present invention may be embodied as a system, a method, and/or a computer program product. The computer program product may include a computer readable storage medium (or media) having computer readable program instructions thereon for causing a processor to carry out aspects of the present invention.

The medium may be an electronic, magnetic, optical, electromagnetic, infrared or a semi-conductor system for a propagation medium. Examples of a computer-readable medium may include a semi-conductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W), DVD and Blu-Ray-Disk.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disk read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Smalltalk, C++ or the like, and conventional procedural programming languages, such as the “C” programming language or similar programming languages. The computer readable program instructions may execute entirely on the user's computer, partly on the user's computer as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatuses, or another device to cause a series of operational steps to be performed on the computer, other programmable apparatus or other device to produce a computer implemented process, such that the instructions which execute on the computer, other programmable apparatuses, or another device implement the functions/acts specified in the flowchart and/or block diagram block or blocks.

The flowcharts and/or block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or act or carry out combinations of special purpose hardware and computer instructions.

The terminology used herein is for the purpose of describing particular embodiments only and is not intended to limit the invention. As used herein, the singular forms “a”, “an” and “the” are intended to include the plural forms as well, unless the context clearly indicates otherwise. It will further be understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or steps plus function elements in the claims below are intended to include any structure, material, or act for performing the function in combination with other claimed elements, as specifically claimed. The description of the present invention has been presented for purposes of illustration and description, but is not intended to be exhaustive or limited to the invention in the form disclosed. Many modifications and variations will be apparent to those of ordinary skills in the art without departing from the scope and spirit of the invention. The embodiments are chosen and described in order to best explain the principles of the invention and the practical application, and to enable others of ordinary skills in the art to understand the invention for various embodiments with various modifications, as are suited to the particular use contemplated.

What is claimed is:

1. A computer-implemented method for assigning medical codes, the method comprising:
receiving a medical record comprising a diagnosis code;
querying a knowledge graph using the diagnosis code, the knowledge graph comprising as nodes: case identifiers, diagnosis codes and related procedure codes, and secondary diagnosis codes and related secondary procedure codes, wherein edges between the nodes are indicative of a type of relationship between related nodes derived from real past medical records; and
receiving, based on the query, a ranked list of the diagnosis codes, related procedure codes, and secondary diagnosis codes and the related secondary procedure codes based on relative occurrences of the past medical records.

2. The method according to claim 1, further comprising:
scanning the medical record and extracting the diagnosis code using optical character recognition.

3. The method according to claim 1, wherein the medical record further comprises:
a secondary diagnosis code with a related secondary procedure code.

4. The method according to claim 2, further comprising:
extracting a procedure code relating to the diagnosis code and at least one secondary diagnosis code with the related secondary procedure code from the scanned medical records, using natural language processing.

5. The method according to claim 1, wherein the knowledge graph further comprises as nodes one or more of an age, gender information, or length of stay.

6. The method according to claim 1, further comprising:
displaying the ranked list; and
receiving a confirmation signal for a selected combination of the diagnosis code and related procedure codes.

7. The method according to claim 6, further comprising:
receiving a confirmation signal for a selected combination of at least one second diagnosis code and one or more related procedure codes.

8. The method according to claim 1, further comprising:
building the knowledge graph from one or more of the real past medical records, a catalogue of the diagnosis codes and the procedure codes, and rules for dependencies between the diagnostic codes and the procedure codes.

9. The method according to claim 1, further comprising:
upon determining in the ranked list that a predefined percentage of a selected diagnosis code relates to a specific procedure code, directly confirming a relationship between the diagnosis code and the procedure code.

10. The method according to claim 1, wherein the diagnosis code is a plurality of diagnoses codes.

11. The method according to claim 10, further comprising:
determining an evidence factor value indicative of a probability for one or more of a main diagnosis code, any other diagnosis code, a main procedure code, and any other procedure code using natural language processing.

12. A computer system for assigning medical codes, the computer system comprising:
one or more computer processors, one or more computer-readable storage media, and program instructions stored on the one or more of the computer-readable storage media for execution by at least one of the one or more processors capable of performing a method, the method comprising:
receiving a medical record comprising a diagnosis code;
querying a knowledge graph using the diagnosis code, the knowledge graph comprising as nodes: case identifiers, diagnosis codes and related procedure codes, and secondary diagnosis codes and related secondary procedure codes, wherein edges between the nodes are indicative of a type of relationship between related nodes derived from real past medical records; and
receiving, based on the query, a ranked list of the diagnosis codes, related procedure codes, and secondary diagnosis codes and the related secondary procedure codes based on relative occurrences of the past medical records.

13. The system according to claim 12, further comprising:
scanning the medical record and extracting the diagnosis code using optical character recognition.

14. The system according to claim 12, wherein the medical record further comprises:
a secondary diagnosis code with a related secondary procedure code.

15. The system according to claim 13, further comprising:
extracting a procedure code relating to the diagnosis code and at least one secondary diagnosis code with the related secondary procedure code from the scanned medical records, using natural language processing.

16. The system according to claim 12, wherein the knowledge graph further comprises as nodes one or more of an age, gender information, or length of stay.

17. The system according to claim 12, further comprising:
displaying the ranked list; and
receiving a confirmation signal for a selected combination of the diagnosis code and related procedure codes.

18. The system according to claim 17, further comprising:
receiving a confirmation signal for a selected combination of at least one second diagnosis code and one or more related procedure codes.

19. The system according to claim 12, further comprising:
building the knowledge graph from one or more of the real past medical records, a catalogue of the diagnosis codes and the procedure codes, and rules for dependencies between the diagnostic codes and the procedure codes.

20. A computer program product for assigning medical codes, the computer program product comprising:
one or more non-transitory computer-readable storage media and program instructions stored on the one or more non-transitory computer-readable storage media capable of performing a method, the method comprising:
receiving a medical record comprising a diagnosis code;
querying a knowledge graph using the diagnosis code, the knowledge graph comprising as nodes: case identifiers, diagnosis codes and related procedure codes, and secondary diagnosis codes and related secondary procedure codes, wherein edges between the nodes are indicative of a type of relationship between related nodes derived from real past medical records; and
receiving, based on the query, a ranked list of the diagnosis codes, related procedure codes, and secondary diagnosis codes and the related secondary procedure codes based on relative occurrences of the past medical records.
